(19) United States
(12) Patent Application Publication
(10) Pub. No.: US 2025/0003004 A1
(43) Pub. Date: Jan. 2, 2025

(54) Personalized Longitudinal Analysis of Circulating Material to Monitor and Adapt Neoantigen Cancer Vaccines

(71) Applicant: AMAZON TECHNOLOGIES, INC., RENO, NV (US)

(72) Inventors: Haibao Tang, Cupertino, CA (US); David Heckerman, Bellevue, WA (US); Layne Christopher Price, Seattle, WA (US); Frank Wilhelm Schmitz, Seattle, WA (US); Alena Harley, Los Gatos, CA (US); Antje Heit, Seattle, WA (US); Samuel Anthony Danziger, Seattle, WA (US)

(21) Appl. No.: 18/271,540

(22) PCT Filed: Jul. 6, 2023
(86) PCT No.: PCT/US2023/069673

§ 371 Date: Jul. 10, 2023

Related U.S. Application Data
(60) Provisional application No. 63/389,384, filed on Jul. 15, 2022.
(60) Provisional application No. 63/380,286, filed on Oct. 20, 2022.

(51) Int. Cl. C12Q 1/6886; A61K 39/00

(52) U.S. Cl. CPC C12Q 1/6886; A61K 39/0011; C12Q 2600/156

(57) ABSTRACT

Disclosed herein is a method, comprising administering to a subject in need thereof an initial immunogenic composition comprising a plurality of tumor-specific neoantigens, each corresponding to a member of a first set of tumor-associated mutations in a subject, and none corresponding to a member of a second set of tumor-associated mutations in the subject; and quantifying each member of the first set of tumor-associated mutations and each member of the second set of tumor-associated mutations in circulating material comprising tumor-associated mutations isolated from the subject at each of multiple time points.

### CROSS-REFERENCE TO RELATED APPLICATIONS

The present application claims the benefit of U.S. Provisional Applications Nos. 63/389,384 and 63/380,286, respectively filed on Jul. 15, 2022, and Oct. 20, 2022, the entire contents of which are incorporated herein by reference.

### 1. BACKGROUND

Cancer is a leading cause of death worldwide accounting for 1 in 4 of all deaths. Siegel et al., CA: A Cancer Journal for Clinicians, 68:7-30 (2018). There were 18.1 million new cancer cases and 9.6 million cancer-related deaths in 2018. Bray et al., CA: A Cancer Journal for Clinicians, 68(6):394-424. There are a number of existing standard of care cancer therapies, including ablation techniques (e.g., surgical procedures and radiation) and chemical techniques (e.g., chemotherapeutic agents). Unfortunately, such therapies are frequently associated with serious risk, toxic side effects, and extremely high costs, as well as uncertain efficacy.

Cancer immunotherapy (e.g., cancer vaccine) has emerged as a promising cancer treatment modality. The goal of cancer immunotherapy is to harness the immune system for selective destruction of cancer while leaving normal tissues unharmed. Traditional cancer vaccines typically target tumor-associated antigens. Tumor-associated antigens are typically present in normal tissues, but overexpressed in cancer. However, because these antigens are often present in normal tissues immune tolerance can prevent immune activation. Several clinical trials targeting tumor-associated antigens have failed to demonstrate a durable beneficial effect compared to standard of care treatment. Li et al., Ann Oncol., 28 (Suppl 12): xii11-xii17 (2017).

Neoantigens represent an attractive target for cancer immunotherapies. Neoantigens are non-autologous proteins with individual specificity. Neoantigens are derived from random somatic mutations in the tumor cell genome and are not expressed on the surface of normal cells. Id. Because neoantigens are expressed exclusively on tumor cells, and thus do not induce central immune tolerance, cancer vaccines targeting cancer neoantigens have potential advantages, including decreased central immune tolerance and improved safety profile. Id.

The mutational landscape of cancer is complex and tumor mutations are generally unique to each individual subject. Most somatic mutations detected by sequencing do not result in effective neoantigens. Only a small percentage of mutations in the tumor DNA, or a tumor cell, are transcribed, translated, and processed into a tumor-specific neoantigen with sufficient accuracy to design a vaccine that is likely to be effective. Further, not all neoantigens are immunogenic. In fact, the proportion of T cells spontaneously recognizing endogenous neoantigens is about 1% to 2%. See, Karpanen et al., Front Immunol., 8:1718 (2017). Moreover, the cost and time associated with the manufacture of neoantigen vaccines is significant.

Compounding these challenges, the prediction of immunogenicity of neoantigens is not perfect. This can lead to the overall efficacy of a neoantigen vaccine being lower than desirable.

Accordingly, a need exists for identifying neoantigens that are not effective when included in a neoantigen vaccine. A further need exists for replacing such ineffective neoantigens with effective ones during the course of a subject's treatment vaccination regimen.

### 2. BRIEF DESCRIPTION OF THE DRAWING

FIG. 1 shows a hypothetical model of various aspects of methods disclosed herein.

### 3. DETAILED DESCRIPTION

This disclosure relates to methods for identifying neoantigens that are not effective when included in a neoantigen vaccine. The disclosure also relates to methods for replacing such ineffective neoantigens with effective ones during the course of a subject's treatment regimen.

In some embodiments, this disclosure relates to a method comprising administering to a subject in need thereof an initial immunogenic composition comprising a plurality of tumor-specific neoantigens, wherein each tumor-specific neoantigen corresponds to a member of a first set of tumor-associated mutations in a subject, and no tumor-specific neoantigen corresponds to a member of a second set of tumor-associated mutations in the subject. The method also comprises quantifying each member of the first set of tumor-associated mutations and each member of the second set of tumor-associated mutations in circulating material isolated from the subject at each of multiple time points. The circulating material can be circulating tumor DNA (ctDNA), circulating free DNA (cfDNA), circulating tumor cells (CTCs), circulating tumor proteins, extracellular vesicles, or two or more thereof.

The method can further comprise replacing a tumor-specific neoantigen corresponding to a member of the first set of tumor-associated mutations from the initial immunogenic composition with a replacement tumor-specific neoantigen corresponding to a member of the second set of tumor-associated mutations, to yield a reformulated immunogenic composition, in response to the quantity of the member of the first set of tumor-associated mutations increasing from an earlier time point to a later time point.

The method can further comprise administering the reformulated immunogenic composition to the subject.

The subject can have any type of cancer where the cancerous cells have genetic mutations, including, but not limited to, melanoma, breast cancer, sarcomas, ovarian cancer, prostate cancer, kidney cancer, gastric cancer, colon cancer, testicular cancer, head and neck cancer, pancreatic cancer, brain cancer, bone cancer, B-cell lymphoma, acute myelogenous leukemia, chronic myelogenous leukemia, chronic lymphocytic leukemia, T-cell lymphocytic leukemia, colon cancer, urothelial cancer, or lung cancer, among others.

The tumor-associated mutations can comprise at least one mutation specific to the subject.

The tumor-associated mutations can comprise at least one tumor hotspot mutation.

The tumor can be ER+/HER2− breast cancer and the at least one tumor hotspot mutation can be in a gene selected from the group consisting of AKT1, APC, ARIDTA, ATM, BRAF, BRCA1, BRCA2, CDH1, CDKN2A, ESR1, GATA3, GNAS, HER2, KRAS, NF1, PIK3CA, PTEN, RB1, SMAD4, and TP53.

The tumor can be melanoma.

In some embodiments, each tumor-specific neoantigen corresponding to a member of the first set of tumor-associated mutations can have a higher immunogenicity score than any tumor-specific neoantigen corresponding to a member of the second set of tumor-associated mutations.

At least one of the multiple time points can be before the initial immunogenic composition is administered.

The initial immunogenic composition can be administered multiple times prior to the replacing of the tumor-specific neoantigen.

The reformulated immunogenic composition can be administered multiple times after the replacing of the tumor-specific neoantigen.

In some embodiments, the quantity of the member of the second set of tumor-associated mutations to which the replacement tumor-specific neoantigen corresponds can remain the same or increase (i.e., not decrease) from the earlier time point to the later time point.

Quantifying the tumor-associated mutations can comprise various methods depending on the circulating material from which the tumor-associated mutations are quantified. Quantifying the tumor-associated mutations can comprise sequencing ctDNA using whole exome sequencing (WES), whole genome sequencing (WGS), targeted sequencing, polymerase chain reaction (PCR), or hybridization methods; sequencing cfDNA using quantitative polymerase chain reaction (qPCR) or next generation sequencing; assaying methylation or chromatin content of ctDNA, cfDNA, or DNA from CTCs; performing mass spectrometry or elution assays on circulating tumor proteins, proteins from CTCs, or proteins from extracellular vesicles; performing fluorescence-activated cell sorting (FACS) on CTCs; or sequencing nucleic acids from CTCs or extracellular vesicles using WES, WGS, targeting sequencing, PCR, qPCR, next generation sequencing, single-cell RNA sequencing, or hybridization methods.

One of the multiple time points can be at least about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks after administering the initial immunogenic composition or the reformulated immunogenic composition.

One of the multiple time points can be at least about 1 month, about 2 months, about 3 months, about 4 months, about 5 months, about 6 months, about 7 months, about 8 months, about 9 months, about 10 months, about 11 months, or about 12 months after administering the initial immunogenic composition or the reformulated immunogenic composition.

One of the multiple time points can be at least about 1 year, about 2 years, about 3 years, about 4 years, about 5 years, about 6 years, about 7 years, about 8 years, about 9 years, or about 10 years, after administering the initial immunogenic composition or the reformulated immunogenic composition.

The circulating material can be isolated from a blood sample, a serum sample, a plasma sample, a urine sample, or a cerebrospinal fluid sample.

The circulating material can be isolated from at least about 10 ml of the subject's whole blood.

The circulating material can be isolated from at least about 20 ml of the subject's whole blood.

The method can further comprise detecting the emergence at a first time point of at least one tumor-associated mutation not included in the first set of tumor-associated mutations or the second set of tumor-associated mutations.

The method can also further comprise adding the emerged tumor-associated mutation to the second set of tumor-associated mutations at a time point after the first time point.

All publications and patents cited in this disclosure are incorporated by reference in their entirety. To the extent the material incorporated by reference contradicts or is inconsistent with this specification, the specification will supersede any such material. The citation of any references herein is not an admission that such references are prior art to the present disclosure. When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise.

Various terms relating to aspects of the description are used throughout the specification and claims. Such terms are to be given their ordinary meaning in the art unless otherwise indicated. Other specifically defined terms are to be construed in a manner consistent with the definitions provided herein. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include plural forms unless the context clearly indicates otherwise. The terms “include,” “such as,” and the like are intended to convey inclusion without limitation, unless otherwise specifically indicated.

Unless otherwise indicated, the terms “at least,” “less than,” and “about,” or similar terms preceding a series of elements or a range are to be understood to refer to every element in the series or range. Those skilled in the art will recognize, or be able to ascertain using no more than routine experimentation, many equivalents to the specific embodiments of the invention described herein. Such equivalents are intended to be encompassed by the following claims.

The term “cancer” refers to the physiological condition in subjects in which a population of cells is characterized by uncontrolled proliferation, immortality, metastatic potential, rapid growth and proliferation rate, and/or certain morphological features. Often cancers can be in the form of a tumor or mass, but may exist alone within the subject, or may circulate in the blood stream as independent cells, such as leukemic or lymphoma cells. The term cancer includes all types of cancers and metastases, including hematological malignancy, solid tumors, sarcomas, carcinomas, and other solid and non-solid tumors. Examples of cancers include, but are not limited to, carcinoma, lymphoma, blastoma, sarcoma, and leukemia. More particular examples of such cancers include squamous cell cancer, small cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer (e.g., triple negative breast cancer, hormone receptor positive breast cancer), osteosarcoma, melanoma, colon cancer, colorectal cancer, endometrial (e.g., serous) or uterine cancer, salivary gland carcinoma, kidney cancer, liver cancer, prostate cancer, vulvar cancer, thyroid cancer, hepatic carcinoma, and various types of head and neck cancers.

The term “subject” as used herein refers to any animal, such as any mammal, including but not limited to, humans, non-human primates, rodents, mammals commonly kept as pets (e.g., dogs and cats, among others), livestock (e.g., cattle, sheep, goats, pigs, horses, and camels, among others) and the like. In some embodiments, the mammal is a mouse. In some embodiments, the mammal is a human.

The term “tumor cell” as used herein refers to any cell that is a cancer cell or is derived from a cancer cell. The term “tumor cell” can also refer to a cell that exhibits cancer-like properties, e.g., uncontrollable reproduction, resistance to anti-growth signals, ability to metastasize, and loss of ability to undergo programed cell death.

Additional description of the methods and guidance for the practice of the methods are provided herein.

### A. Tumor-Associated Mutations and Corresponding Tumor-Specific Neoantigens

DNA from tumors generally exhibits one or more mutations relative to DNA from normal or healthy tissue. Some of these tumor-associated mutations are relatively prevalent in patient populations. Such relatively prevalent tumor-associated mutations can be termed tumor hotspot mutations. The gene(s) in which tumor hotspot mutations occur will depend on the particular cancer. For example, in ER+/HER2−breast cancer, a tumor hotspot mutation can be in a gene selected from the group consisting of AKT1, APC, ARID1A, ATM, BRAF, BRCA1, BRCA2, CDH1, CDKN2A, ESRI, GATA3, GNAS, HER2, KRAS, NF1, PIK3CA, PTEN, RB1, SMAD4, and TP53. Other tumor hotspot mutations and the genes in which they occur are known in other cancers, such as melanoma.

In some embodiments, the tumor-associated mutations comprise at least one tumor hotspot mutation.

Other tumor-associated mutations can be subject-specific. These particular tumor-associated mutations cannot be predicted in advance, but can only be determined from a workflow comprising sequencing healthy and tumor nucleic acid sequences, such as by whole exome sequencing (WES) or whole genome sequencing (WGS) of DNA from healthy tissue and tumor tissue, by single-cell RNA (scRNA) sequencing of healthy cells or tumor cells, or other techniques. Normal and tumor sequences can then be aligned and compared, with differences between the two types of aligned sequences being identified as tumor-associated mutations.

In WES and scRNA sequencing, the sequenced data is from nucleic acid sequences that are transcribed and translated into peptides. Data from WGS includes nucleic acid sequences that are transcribed and translated into peptides. Hence, any tumor-associated mutation found by WES or scRNA sequencing of tumor nucleic acids will, upon translation, yield a peptide expected to be present in the tumor and expected to be absent from healthy tissue. Such a peptide can be considered a tumor-specific neoantigen. The tumor-specific neoantigen can be considered as corresponding to the tumor-associated mutation. Similarly, at least some tumor-associated mutations found by WGS sequencing of tumor DNA will have a tumor-specific neoantigen corresponding thereto.

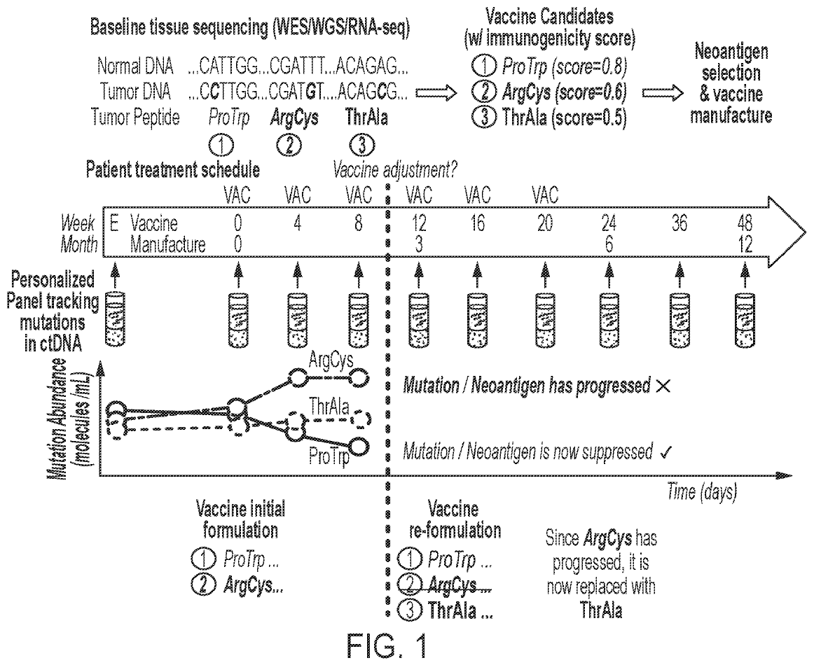

FIG. 1 presents a hypothetical model to aid in understanding. At the upper left, WES, WGS, or scRNA sequencing (RNA-seq) are performed on normal tissue and healthy tissue. Sequenced data is aligned and three tumor-associated mutations are shown: CATTGG→CCTTGG, CGATTT→CGATGT, and ACAGAG→ACAGCG. Each of these three tumor-associated mutations has a tumor-specific neoantigen corresponding thereto: peptide 1, comprising ProTrp; peptide 2, comprising ArgCys; peptide 3, comprising ThrAla. In practice, greater numbers of tumor-associated mutations and longer tumor-specific neoantigen sequences will be found.

Two factors should be borne in mind when determining subject-specific tumor-associated mutations. One is that after the initial determination, subclonal mutations may arise, which can allow a tumor to evolve resistance to an immunogenic composition not targeting a tumor-specific neoantigen corresponding to such a subclonal mutation. Second, clonal hematopoiesis of indeterminate potential (CHIP) is the constant accumulation with age of mutations in white blood cells. This latter factor can be mitigated at least in part by performing additional sequence analysis of white blood cells to exclude CHIP-associated variants.

In some embodiments, the tumor-associated mutations comprise at least one mutation specific to the subject.

### B. Immunogenic Compositions

Once polypeptide sequence data of one or more tumor-specific neoantigens is obtained, a numerical probability score can be generated that forecasts whether the one or more tumor-specific neoantigens are immunogenic (e.g., will elicit an immune response in the subject). For example, the polypeptide sequence data and the MHC molecule can be inputted into a machine-learning platform (i.e., model(s)). The machine-learning platform can generate the numerical probability score.

MHC molecules transport and present peptides on the cell surface. The MHC molecules are classified as MHC molecules of class I and of class IL. MHC class I molecules are present on the surface of almost all cells of the body, including most tumor cells. The proteins of MHC class I are loaded with antigens that usually originate from endogenous proteins or from pathogens present inside cells, and are then presented to cytotoxic T-lymphocytes (i.e., CD8+). The MHC class I molecules can comprise HLA-A, HLA-B, or HLA-C. The MHC molecules of class II are only present on dendritic cells, B lymphocytes, macrophages, and other antigen-presenting cells. They present mainly peptides, which are processed from external antigen sources, i.e. outside of the cells, to T-helper (Th) cells (i.e., CD4+). The MHC class II molecules can comprise HLA-DPA1, HLA-DPB1, HLA-DQA1, HLA-DQB1, HLA-DRA, and HLA-DRB1. In some occasions, MHC class II molecules can also be expressed on cancer cells.

MHC class T molecules and/or MHC class TT molecules can be inputted into a machine-learning platform. Typically, either MHC class I molecules or MHC class II molecules are inputted into a machine-learning platform. In some embodiments, MHC class I molecules are inputted into a machine-leaming platform. In other embodiments, MHC class II molecules are inputted into a machine-learning platform. In some embodiments, an MHC class I machine-learning platform may be trained on MHC class I training data. In some embodiments, an MHC class II machine-learning platform may be trained on MHC class II training data. In some embodiments the same machine-learning platform may be trained on both MHC class I and class II training data. In some embodiments, the machine-learning platform may include an MHC class I model and an MHC class II model.

MHC class I molecules bind to short peptides. MHC class I molecules can accommodate peptides generally about 8 amino acids to about 10 amino acids in length. In embodiments, the sequence data encoding one or more tumor-specific neoantigens are short peptides about 8 amino acids to about 10 amino acids in length. MHC class II molecules bind to peptides that are longer in length. MHC class II can accommodate peptides which are generally about 13 amino acids in length to about 25 amino acids in length. In embodiments, the sequence data encoding one or more tumor-specific neoantigens are long peptides about 13 to 25 amino acids in length.

The sequence data encoding one or more tumor-specific neoantigens can be about 5 amino acids in length, about 6 amino acids in length, about 7 amino acids in length, about 8 amino acids in length, about 9 amino acids in length, about 10 amino acids in length, about 11 amino acids in length, about 12 amino acids in length, about 13 amino acids in length, about 14 amino acids in length, about 15 amino acids in length, about 16 amino acids in length, about 17 amino acids in length, about 18 amino acids in length, about 19 amino acids in length, about 20 amino acids in length, about 21 amino acids in length, about 22 amino acids in length, about 23 amino acids in length, about 24 amino acids in length, about 25 amino acids in length, about 26 amino acids in length, about 27 amino acids in length, about 28 amino acids in length, about 29 amino acids in length, or about 30 amino acids in length.

Immunogenic tumor-specific neoantigens are not expressed in normal tissues. They can be presented by antigen-presenting cells to CD4+ and CD8+ T-cells to generate an immune response. In embodiments, an immune response in the subject elicited by the one or more tumor-specific neoantigens comprises presentation of the one or more tumor-specific neoantigens to the tumor cell surface. More specifically, the immune response in the subject elicited by the one or more tumor-specific neoantigens comprises presentation of the one or more tumor-specific neoantigens by one or more MHC molecules on the tumor cell. It is expected that the immune response elicited by the one or more tumor-specific neoantigens is a T-cell mediated response. The immune response in the subject elicited by the one or more tumor-specific neoantigens may involve one or more tumor-specific neoantigens being capable of presentation to T-cells by antigen presenting cells, such as dendritic cells. In one aspect, the one or more tumor-specific neoantigens is capable of activating CD8+ T-cells and/or CD4+ T-cells.

In embodiments, a machine-learning platform can predict the likelihood the one or more tumor-specific neoantigens will activate CD8+ T cells. In embodiments, a machine learning platform can predict the likelihood that one or more tumor-specific neoantigens will activate CD4+ T cells. In some instances, a machine-learning platform can predict the antibody titer that one or more tumor-specific neoantigens can elicit. In other instances, a machine-learning platform can predict the frequency of CD8+ activation by one or more tumor-specific neoantigens.

A machine-learning platform can include a model trained on training data. Training data can be obtained from a series of distinct subjects. The training data can comprise data derived from healthy subjects, as well as subjects having cancer. The training data may include various data that can be used to generate a probability score that indicates whether the one or more tumor-specific neoantigens will elicit an immune response in a subject. Exemplary training data can include data representing nucleotide or polypeptide sequences derived from normal tissue and/or cells, data representing nucleotide or polypeptide sequences derived from tumor tissue, data representing MHC peptidome sequences from normal and tumor tissue, peptide-MHC binding affinity measurement, or combinations thereof. The training data can further comprise mass spectrometry data, DNA sequencing data, RNA sequencing data, clinical data from healthy subjects and subjects having cancer, cytokine profiling data, T cell cytotoxicity assay data, peptide-MHC mono-or-multimer data, and proteomics data for single-allele cell lines engineered to express a predetermined MHC allele that are subsequently exposed to synthetic protein, normal and tumor human cell lines, fresh and frozen primary samples, and T-cell assays.

A machine-learning platform can be a supervised learning platform, an unsupervised learning platform, or a semi-supervised learning platform. A machine-learning platform can use sequence-based approach to generate a numerical probability that the one or more tumor-specific neoantigens can elicit an immune response (e.g., will induce a high or low antibody response or CD8+ response). Sequence based predictions can include supervised machine-learning modules including artificial neural networks (e.g., deep or otherwise), support vector machines, K-nearest neighbor, Logistic Multiple Network-constrained Regression (LogMiNeR), regression tree, random forest, adaBoost, XGBoost, or hidden Markov models. These platforms require training data sets that include known MHC binding peptides.

Numerous prediction programs have been employed to predict whether a tumor-specific neoantigen can be presented on an MHC molecule and elicit an immune response. Exemplary predictive programs include, for example, HLAminer (Warren et al., Genome Med., 4:95 (2012); HLA type predicted by orienting the assembly of shotgun sequence data and comparing it with the reference allele sequence database), The Ensembl Variant Effect Predictor (McLaren et al., Genome Biol., 17:122 (2016)), NetMHCpan (Andreatta et al., Bioinformatics, 32:511-517 (2016); sequence comparison method based on artificial neural network, and predict the affinity of peptide-MHC—I type molecular), UCSC browser (Kent et al., Genome Res., 12:996-1006 (2002)), CloudNeo pipeline (Bais et al., Bioinformatics, 33:3110-2 (2017)), OptiType (Szolek et al., Bioinformatics, 30:3310-3316 (2014)), ATHLATES (Liu C et al., Nucleic Acids Res. 41:e142 (2013)), pVAC-Seq (Hundal et al., Genome Med. 8:11 (2016)), MuPeXI (Bjerregaard et al., Cancer Immunol. Immunother., 66:1123-30 (2017)), Strelka (Saunders et al., Bioinformatics, 28:1811-7 (2012)), Strelka2 (Kim et al., Nat Methods. 15:591-4 (2018)), VarScan2 (Koboldt et al., Genome Res., 22:568-76 (2012)), SomaticSeq (Fang L et al., Genome Biol., 16:197 (2015)), SMMPMBEC (Kim et al., BMC Bioinformatics, 10:394 (2009)), NeoPredPipe (Schenck RO, BMC Bioinformatics, 20:264 (2019)), Weka (Witten et al., Data mining: practical machine-learning tools and techniques. 4^{th} ed. Elsevier, ISBN: 97801280435578 (eBook) (2017)), or Orange (Demsar et al., Orange: Data Mining Toolbox in Python., J. Mach. Learn. Res., 14:2349-2353 (2013)). Any known predictive programs may be employed as the machine-learning platform to generate a numerical probability score that indicates whether the neoantigen will elicit an immune response.

Depending on the machine-learning platform employed, additional filters can be applied to prioritize tumor-specific neoantigen candidates, including elimination of hypothetical (Riken) proteins; use of an antigen processing algorithm to eliminate epitopes that are not likely to be proteolytically produced by the constitutive- or immune-proteasome and prioritization of neoantigens where the neoantigen has a higher predicted binding affinity than the corresponding wildtype sequence.

The numerical probability score can be a number between 0 and 1. In embodiments, the numerical probability score can be a number of 0, 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, or 1. A tumor-specific neoantigen with a higher numerical probability score relative to a lower numerical probability score indicates that the tumor-specific neoantigen will elicit a greater immune response in the subject, and thus is likely to be a suitable candidate for an immunogenic composition. For example, a tumor-specific neoantigen with a numerical probability score of 1 will likely elicit a greater immune response in a subject than a tumor-specific neoantigen having a numerical probability score of 0.05. Similarly, a tumor-specific neoantigen having a numerical probability score of 0.5 will likely elicit a greater immune response in a subject than a tumor-specific neoantigen with a numerical probability score of 0.1.

A higher numerical probability score relative to a lower numerical probability score is preferable. Preferably, a tumor-specific neoantigen having a numerical probability score of at least 0.80, 0.81, 0.82. 0.83, 0.84, 0.85, 0.86, 0.87, 0.88, 0.89, 0.90, 0.95, 0.96, 0.97, 0.98, 0.99, or 1 indicates that an immune response will likely be elicited in the subject.

While a higher numerical probability score is preferable, a lower numerical probability score may still indicate that the tumor-specific neoantigen is capable of eliciting a sufficient immune response, such that the tumor-specific neoantigen is likely to be a suitable candidate.

In instances, the machine-learning platform can also predict the likelihood that the one or more tumor-specific neoantigens will be presented by an MHC molecule on a tumor cell. The machine-learning platform can predict the likelihood that one or more tumor-specific neoantigens will be presented by an MHC class I molecule or MHC class II molecule.

The methods for selecting one or more tumor-specific neoantigens may further comprise a step of measuring, in silico, the affinity of one or more tumor-specific neoantigens to bind to an MHC molecule in the subject. A tumor-specific neoantigen that has a binding affinity with an MHC molecule of less than about 1000 nM indicates that the one or more tumor-specific neoantigens may be suitable for an immunogenic composition. A tumor-specific neoantigen that has a binding affinity with a MHC molecule of less than about 500 nM, of less than about 400 nM, of less than about 300 nM, of less than about 200 nM, of less than about 100 nM, or of less than about 50 nM can indicate that one or more tumor-specific neoantigens may be suitable for an immunogenic composition. The affinity of the one or more tumor-specific neoantigens to bind to an MHC molecule in the subject can predict tumor-specific neoantigen immunogenicity. Alternatively, median affinity can be an effective way to predict tumor-specific neoantigen immunogenicity. Median affinity can be calculated using epitope prediction algorithms, such as NetMHCpan, Artificial Neural Networks (ANN), Stabilized Matrix Method (SMM), and SMMPMBEC.

RNA expression of one or more tumor-specific neoantigens can also be quantified. RNA expression of one or more tumor-specific neoantigens can be quantified to identify one or more neoantigens that will elicit an immune response in a subject. A variety of methods exist for measuring RNA expression. Known techniques, which may measure RNA expression, include RNA-seq, and in situ hybridization (e.g., FISH), Northern blot, DNA microarray, Tiling array, and quantitative polymerase chain reaction (qPCR). Other known techniques in the art can be used to quantify RNA expression. RNA can be messenger RNA (mRNA), short-interfering RNA (siRNA), microRNA (miRNA), circular RNA (circRNA), transfer RNA (tRNA), ribosomal RNA (rRNA), small nucleolar RNA (snRNA), Piwi-interacting RNA (piRNA), long non-coding RNA (long ncRNA), sub-genomic RNA (sgRNA), RNA from integrating or non-integrating viruses, or any other RNA. In one aspect, mRNA expression is measured.

The present technique can further reduce the likelihood of selecting tumor-specific neoantigen may induce an autoimmune response in normal tissues. It is expected that a tumor-specific neoantigen that has similar sequence to a normal antigen may induce an autoimmune response in normal tissue. For example, a tumor-specific neoantigen that is at least 90%, at least 91%, at least 92%, at least 93%, at least 94%, at least 95%, at least 96%, at least 97%, at least 98%, or at least 99% similar to a normal antigen may induce an autoimmune response. Tumor-specific neoantigens that are predicted to induce an autoimmune response are not prioritized for the immunogenic composition. Tumor-specific neoantigens that are predicted to induce an autoimmune response are typically not selected for the immunogenic composition. The method can further comprise measuring the ability of the one or more tumor-specific neoantigen to invoke immunological tolerance. Tumor-specific neoantigens that are predicted to invoke immunological tolerance are not prioritized for the immunogenic composition.

The tumor sub-clone profile can be used to inform selection of tumor-specific neoantigens for an immunogenic composition. For example, for any tumor sub-clones identified prior to formulation of the initial immunogenic composition, at least one tumor-specific neoantigen associated with each sub-clone may be considered for inclusion in the initial immunogenic composition.

An immunogenicity score can be determined for each tumor-specific neoantigen based on one or more of the numerical probability score, the likelihood of activation of CD8+ T cells, the predicted antibody titer, one or more of the additional filters, the likelihood of presentation by an MHC class I molecule or an MHC class II molecule, the affinity of binding with an MHC molecule, RNA quantification, or the likelihood of an autoimmune response.

In view of the immunogenicity score of each tumor-specific neoantigen, one or more tumor-specific neoantigens can be selected for formulation of a subject-specific initial immunogenic composition. The selection can be for any tumor-specific neoantigens having an immunogenicity score at or above a desired value, or for the n-highest tumor-specific neoantigens ranked by immunogenicity score. In some embodiments, each tumor-specific neoantigen corresponding to a member of the first set of tumor-associated mutations has a higher immunogenicity score than any tumor-specific neoantigen corresponding to a member of the second set of tumor-associated mutations.

In embodiments, at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 25, at least about 30, at least about 35, at least about 40, at least about 50 or more tumor-specific neoantigens are selected for the initial immunogenic composition. Typically, at least about 10 tumor-specific neoantigens are selected. In other instances, at least about 20 tumor-specific neoantigens are selected.

The immunogenic composition can further comprise natural or synthetic antigens. The natural or synthetic antigens can increase the immune response. Exemplary natural or synthetic antigens include, but are not limited to, pan-DR epitope (PADRE) and tetanus toxin antigen.

The immunogenic composition can be in any form, for example a synthetic long peptide, RNA, DNA, a cell, a dendritic cell, a nucleotide sequence, a polypeptide sequence, a plasmid, or a vector.

Tumor-specific neoantigens can also be included in viral vector-based vaccine platforms, such as vaccinia, fowlpox, self-replicating alphavirus, Maraba virus, adenovirus (See, e.g., Tatsis et al., Molecular Therapy, 10:616-629 (2004)), or lentivirus, including but not limited to second, third or hybrid second/third generation lentivirus and recombinant lentivirus of any generation designed to target specific cell types or receptors (See, e.g., Hu et al., Immunol Rev., 239(1): 45-61 (2011); Sakma et al, Biochem J., 443(3):603-18 (2012)). Dependent on the packaging capacity of the above-mentioned viral vector-based vaccine platforms, this approach can deliver one or more nucleotide sequences that encode one or more tumor-specific neoantigen peptides. The sequences may be flanked by non-mutated sequences, may be separated by linkers, or may be preceded with one or more sequences targeting a subcellular compartment (See, e.g., Gros et al., Nat Med., 22 (4):433-8 (2016); Stronen et al., Science., 352(6291): 1337-1341 (2016); Lu et al., Clin Cancer Res., 20(13):3401-3410 (2014)). Upon introduction into a host, infected cells express the one or more tumor-specific neoantigens, and thereby elicit a host immune (e.g., CD8+ or CD4+) response against the one or more tumor-specific neoantigens. Vaccinia vectors and methods useful in immunization protocols are described in, e.g., U.S. Pat. No. 4,722,848. Another vector is BCG (Bacillus Calmette Guerin). BCG vectors are described in Stover et al. (Nature 351:456-460 (1991)). A wide variety of other vaccine vectors useful for therapeutic administration or immunization of neoantigens that will be apparent to those skilled in the art from the description herein may also be used.

The immunogenic composition can contain individualized components, according to the personal needs of the particular subject.

The immunogenic composition described herein can further comprise an adjuvant. Adjuvants are any substance whose admixture into an immunogenic composition increases, or otherwise enhances and/or boosts the immune response to a tumor-specific neoantigen, but when the substance is administered alone does not generate an immune response to a tumor-specific neoantigen. The adjuvant preferably generates an immune response to the neoantigen and does not produce an allergy or other adverse reaction. It is contemplated herein that the immunogenic composition can be administered before, together, concomitantly with, or after administration of the immunogenic composition.

Adjuvants can enhance an immune response by several mechanisms including, e.g., lymphocyte recruitment, stimulation of B and/or T cells, and stimulation of macrophages. When an immunogenic composition of the invention comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum salts (alum) (such as aluminum hydroxide, aluminum phosphate, and aluminum sulfate), 3 De-O-acylated monophosphoryl lipid A (MPL) (see, GB 2220211), MF59 (Novartis), ASO3 (Glaxo SmithKline), ASO4 (Glaxo SmithKline), polysorbate 80 (Tween 80; ICL Americas, Inc.), imidazopyridine compounds (see, International Application No. PCT/US2007/064857, published as International Publication No. WO2007/109812), imidazoquinoxaline compounds (see, International Application No. PCT/US2007/064858, published as International Publication No. WO2007/109813) and saponins, such as QS21 (see, Kensil et al, in Vaccine Design: The Subunit and Adjuvant Approach (eds. Powell & Newman, Plenum Press, NY, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see, Stoute et al, N. Engl. J. Med. 336, 86-91 (1997)).

CpG immunostimulatory oligonucleotides have also been reported to enhance the effects of adjuvants in a vaccine setting. Other TLR binding molecules such as RNA binding TLR 7, TLR 8 and/or TLR 9 may also be used.

Other examples of useful adjuvants include, but are not limited to, chemically modified CpGs (e.g. CpR, Idera), Poly(I:C)(e.g. polyi:CI2U), poly ICLC, non-CpG bacterial DNA or RNA as well as immunoactive small molecules and antibodies such as cyclophosphamide, sunitinib, bevacizumab, Celebrex (celecoxib), NCX-4016, sildenafil, tadalafil, vardenafil, sorafenib, XL-999, CP-547632, pazopanib, ZD2171, AZD2171, ipilimumab, tremelimumab, and SC58175, which may act therapeutically and/or as an adjuvant. In embodiments, Poly ICLC is an adjuvant.

The immunogenic compositions can comprise one or more tumor-specific neoantigens described herein alone or together with a pharmaceutically acceptable carrier. Suspensions or dispersions of one or more tumor-specific neoantigens, especially isotonic aqueous suspensions, dispersions, or amphiphilic solvents can be used. The immunogenic compositions may be sterilized and/or may comprise excipients, e.g., preservatives, stabilizers, wetting agents and/or emulsifiers, solubilizers, salts for regulating osmotic pressure and/or buffers and are prepared in a manner known per se, for example by means of conventional dispersing and suspending processes. In certain embodiments, such dispersions or suspensions may comprise viscosity-regulating agents. The suspensions or dispersions are kept at temperatures around 2° C. to 8° C., or preferentially for longer storage may be frozen and then thawed shortly before use. For injection, the vaccine or immunogenic preparations may be formulated in aqueous solutions, preferably in physiologically compatible buffers such as Hanks's solution, Ringer's solution, or physiological saline buffer. The solution may contain formulatory agents such as suspending, stabilizing and/or dispersing agents.

In certain embodiments, the compositions described herein additionally comprise a preservative, e.g., the mercury derivative thimerosal. In a specific embodiment, the pharmaceutical compositions described herein comprise 0.001% to 0.01% thimerosal. In other embodiments, the pharmaceutical compositions described herein do not comprise a preservative.

An excipient can be present independently of an adjuvant. The function of an excipient can be, for example, to increase the molecular weight of the immunogenic composition, to increase activity or immunogenicity, to confer stability, to increase the biological activity, or to increase serum-half life. An excipient can also be used to aid presentation of the one or more tumor-specific neoantigens to T-cells (e.g., CD4+ or CD8+ T-cells). The excipient can be a carrier protein such as, but not limited to, keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier is generally a physiologically acceptable carrier acceptable to humans and safe. Alternatively, the carrier can be dextran, for example Sepharose.

Cytotoxic T-cells recognize an antigen in the form of a peptide bound to an MHC molecule, rather than the intact foreign antigen itself. The MHC molecule itself is located at the cell surface of an antigen presenting cell. Thus, an activation of cytotoxic T-cells is possible if a trimeric complex of peptide antigen, MHC molecule, and antigen-presenting cell (APC) is present. The immune response can be enhanced if additional APCs with the respective MHC molecule are added, instead of using only the one or more tumor-specific antigens for activation of cytotoxic T-cells. Thus, in some embodiments an immunogenic composition additionally contains at least one APC.

The immunogenic composition can comprise an acceptable carrier (e.g., an aqueous carrier). A variety of aqueous carriers can be used, e.g., water, buffered water, 0.9% saline, 0.3% glycine, hyaluronic acid and the like. These compositions can be sterilized by conventional, well-known sterilization techniques, or can be sterile filtered. The resulting aqueous solutions can be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

Neoantigens can also be administered via liposomes, which target them to a particular cell tissue, such as lymphoid tissue. Liposomes are also useful in increasing half-life. Liposomes include emulsions, foams, micelles, insoluble monolayers, liquid crystals, phospholipid dispersions, lamellar layers and the like. In these preparations the neoantigen to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired neoantigen can be directed to the site of lymphoid cells, where the liposomes then deliver the selected immunogenic compositions. Liposomes can be formed from standard vesicle-forming lipids, which generally include neutral and negatively charged phospholipids and a sterol, such as cholesterol. The selection of lipids is generally guided by consideration of, e.g., liposome size, acid lability, and stability of the liposomes in the blood stream. A variety of methods are available for preparing liposomes, as described in, e.g., Szoka et al., Annu. Rev. Biophys. Bioeng. 9; 467 (1980), U.S. Pat. Nos. 4,235,871, 4,501,728, 4,501,728, 4,837,028, and 5,019,369.

For targeting to the immune cells, a ligand to be incorporated into the liposome can include, e.g., antibodies or fragments thereof specific for cell surface determinants of the desired immune system cells. A liposome suspension can be administered intravenously, locally, topically, etc. in a dose which varies according to, inter alia, the manner of administration, the peptide being delivered, and the stage of the disease being treated.

As an alternative method for targeting immune cells, components of the immunogenic composition, such as an antigen (i.e., tumor-specific neoantigen), ligand, or adjuvant (e.g., TLR) can be incorporated into a poly(lactic-co-glycolic) microsphere. The poly(lactic-co-glycolic) microspheres can entrap components of the immunogenic composition as an endosomal delivery device.

For therapeutic or immunization purposes, nucleic acids encoding a tumor-specific neoantigen described herein can also be administered to the patient. A number of methods are conveniently used to deliver the nucleic acids to the patient. For example, the nucleic acid can be delivered directly, as “naked DNA.” This approach is described, for example, in Wolff et al., Science 247: 1465-1468 (1990), as well as U.S. Pat. Nos. 5,580,859 and 5,589,466. The nucleic acids can also be administered using ballistic delivery as described, for example, in U.S. Pat. No. 5,204,253. Particles comprised solely of DNA can be administered. Alternatively, DNA can be adhered to particles, such as gold particles. Approaches for delivering nucleic acid sequences can include viral vectors, mRNA vectors, and DNA vectors with or without electroporation. The nucleic acids can also be delivered complexed to cationic compounds, such as cationic lipids.

The immunogenic compositions provided herein can be administered to the subject by various routes including, but not limited to, oral, intradermal, intratumoral, intramuscular, intraperitoneal, intravenous, topical, subcutaneous, percutaneous, intranasal and inhalation routes, and via scarification (scratching through the top layers of skin, e.g., using a bifurcated needle). The immunogenic composition can be administered at the tumor site to induce a local immune response to the tumor.

The dosage of the one or more tumor-specific neoantigens may depend upon the type of composition and upon the subject's age, weight, body surface area, individual condition, the individual pharmacokinetic data, and the mode of administration.

Also disclosed herein is a method of manufacturing an immunogenic composition comprising one or more tumor-specific neoantigens selected by performing the steps of the methods disclosed herein. An immunogenic composition as described herein can be manufactured using methods known in the art. For example, a method of producing a tumor-specific neoantigen or a vector (e.g., a vector including at least one sequence encoding one or more tumor-specific neoantigens) disclosed herein can include culturing a host cell under conditions suitable for expressing the neoantigen or vector, wherein the host cell comprises at least one polynucleotide encoding the neoantigen or vector, and purifying the neoantigen or vector. Standard purification methods include chromatographic techniques, electrophoretic, immunological, precipitation, dialysis, filtration, concentration, and chromatofocusing techniques.

Host cells can include a Chinese Hamster Ovary (CHO) cell, NSO cell, yeast, or a HEK293 cell. Host cells can be transformed with one or more polynucleotides comprising at least one nucleic acid sequence that encodes one or more tumor-specific neoantigens or vector disclosed herein. In certain embodiments the isolated polynucleotide can be cDNA.

Upon selection of tumor-specific neoantigens for the initial immunogenic composition, the tumor-associated mutations can be divided into multiple sets, such as two sets. The first set includes tumor-associated mutations to which a selected tumor-specific neoantigen corresponds. The second set includes tumor-associated mutations to which none of the selected tumor-specific neoantigens corresponds.

FIG. 1 provides a further aid for visualization. At the upper right, the three peptides of the hypothetical model are scored for immunogenicity and ranked: peptide 1, comprising ProTrp, immunogenicity score 0.8; peptide 2, comprising ArgCys, immunogenicity score 0.6; peptide 3, comprising ThrAla, immunogenicity score 0.5. As shown at the lower left, the initial immunogenic composition is selected to include the two highest-ranked tumor-specific neoantigens, peptide 1 and peptide 2. Peptide 3 is excluded from the initial immunogenic composition.

### C. Administration of the Immunogenic Composition to Subjects in Need Thereof

The immunogenic composition described above, comprising tumor-specific neoantigens corresponding to a first set of tumor-associated mutations and not corresponding to a second set of tumor-associated mutations, can be administered to a subject in need thereof, e.g., a subject suffering from or at risk of suffering from cancer.

The cancer can be any solid tumor or any hematological tumor. The tumor can be a primary tumor or a metastasis. Solid tumors can include, but are not limited to, breast cancer tumors, ovarian cancer tumors, prostate cancer tumors, lung cancer tumors, kidney cancer tumors, gastric cancer tumors, testicular cancer tumors, head and neck cancer tumors, pancreatic cancer tumors, brain cancer tumors, and melanoma tumors. Hematological tumors can include, but are not limited to, tumors from lymphomas (e.g., B cell lymphomas) and leukemias (e.g., acute myelogenous leukemia, chronic myelogenous leukemia, chronic lymphocytic leukemia, and T cell lymphocytic leukemia).

The methods disclosed herein can be used for any suitable cancerous tumor, including hematological malignancy, solid tumors, sarcomas, carcinomas, and other solid and non-solid tumors. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor. In one aspect, the cancer is melanoma, breast cancer, ovarian cancer, prostate cancer, kidney cancer, gastric cancer, colon cancer, testicular cancer, head and neck cancer, pancreatic cancer, brain cancer, B-cell lymphoma, acute myelogenous leukemia, chronic myelogenous leukemia, chronic lymphocytic leukemia, T-cell lymphocytic leukemia, bladder cancer, or lung cancer. Melanoma is of particular interest. Breast cancer, lung cancer, and bladder cancer are also of particular interest.

Immunogenic compositions stimulate a subject's immune system, especially the response of specific CD8+ T cells or CD4+ T cells. Interferon gamma produced by CD8+ and T helper CD4+ cells regulate the expression of PD-L1. PD-L1 expression in tumor cells is upregulated when attacked by T cells. Therefore, tumor vaccines may induce the production of specific T cells and simultaneously upregulate the expression of PD-L1, which may limit the efficacy of the immunogenic composition. In addition, while the immune system is activated, the expression of T cell surface reporter CTLA-4 is correspondingly increased, which binds with the ligand B7-1/B7-2 on antigen-presenting cells and plays an immunosuppressant effect. Thus, in some instances, the subject may further be administered an anti-immunosuppressive or immunostimulatory, such as a checkpoint inhibitor. Checkpoint inhibitors can include, but are not limited to, anti-CTL4-A antibodies, anti-PD-1 antibodies and anti-PD-L1 antibodies. These checkpoint inhibitors bind to the immune checkpoint proteins of T cells to remove the inhibition of T cell function by tumor cells. Blockade of CTLA-4 or PD-L1 by antibodies can enhance the immune response to cancerous cells in the patient. CTLA-4 has been shown effective when following a vaccination protocol.

An immunogenic composition comprising one or more tumor-specific neoantigens can be administered to a subject that has been diagnosed with cancer, is already suffering from cancer, has recurrent cancer (i.e., relapse), or is at risk of developing cancer. An immunogenic composition comprising one or more tumor-specific neoantigens can be administered to a subject that is resistant to other forms of cancer treatment (e.g., chemotherapy, immunotherapy, or radiation). An immunogenic composition comprising one or more tumor-specific neoantigens can be administered to the subject prior to other standard of care cancer therapies (e.g., chemotherapy, immunotherapy, or radiation). An immunogenic composition comprising one or more tumor-specific neoantigens can be administered to the subject concurrently, after, or in combination with other standard of care cancer therapies (e.g., chemotherapy, immunotherapy, or radiation).

The subject can be a human, dog, cat, horse, or any animal for which a tumor specific response is desired.

The immunogenic composition is administered to the subject in an amount sufficient to elicit an immune response to the tumor-specific neoantigen and to destroy, or at least partially arrest, symptoms and/or complications. In embodiments, the immunogenic composition can provide a long-lasting immune response. A long-lasting immune response can be established by administering a boosting dose of the immunogenic composition to the subject. The immune response to the immunogenic composition can be extended by administering to the subject a boosting dose. In embodiments, at least one, at least two, at least three or more boosting doses can be administered to abate the cancer. A first boosting dose may increase the immune response by at least 50%, at least 100%, at least 200%, at least 300%, at least 400%, at least 500%, or at least 1000% relative to the initial immune response. A second boosting dose may increase the immune response by at least 50%, at least 100%, at least 200%, at least 300%, at least 400%, at least 500%, or at least 1000% relative to the initial immune response. A third boosting dose may increase the immune response by at least 50%, at least 100%, at least 200%, at least 300%, at least 400%, at least 500%, or at least 1000% relative to the initial immune response.

An amount adequate to elicit an immune response is defined as a “therapeutically effective dose.” Amounts effective for this use will depend on, e.g., the composition, the manner of administration, the stage and severity of the disease being treated, the weight and general state of health of the patient, and the judgment of the prescribing physician. It should be kept in mind that immunogenic compositions can generally be employed in serious disease states, that is, life-threatening or potentially life-threatening situations, especially when the cancer has metastasized. In such cases, in view of the minimization of extraneous substances and the relative nontoxic nature of a neoantigen, it is possible and can be felt desirable by the treating physician to administer substantial excesses of these immunogenic compositions.

The immunogenic composition comprising one or more tumor-specific neoantigens can be administered to the subject alone or in combination with other therapeutic agents. The therapeutic agent can be, for example, a chemotherapeutic agent, radiation, or immunotherapy. Any suitable therapeutic treatment for a particular cancer can be administered. Exemplary chemotherapeutic agents include, but are not limited to aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol®), pilocarpine, prochlorperazine, rituximab, tamoxifen, topotecan hydrochloride, trastuzumab, vinblastine, vincristine, and vinorelbine tartrate. The subject may be administered a small molecule or targeted therapy (e.g., kinase inhibitor). The subject may be further administered an anti-CTLA antibody, anti-PD-1 antibody, or anti-PD-L1 antibody. Blockade of CTLA-4 or PD-L1 by antibodies can enhance the immune response to cancerous cells in the patient.

### D. Circulating Material and Quantification of Tumor-Associated Mutations

Although each of the tumor-specific neoantigens in the initial immunogenic composition is expected to be efficacious against tumors, in reality, it is likely that at least some tumor-specific neoantigens may be ineffective targets for the initial immunogenic composition. Even if all tumor-specific neoantigens are effective, this cannot be predicted in advance.

This disclosure further relates to monitoring, at multiple time points, levels of each tumor-associated mutation in the subject's tumor. If the tumor-associated mutation in the first set of tumor-associated mutations increases over time, the tumor-specific neoantigen corresponding to that tumor-associated mutation can be concluded to be an ineffective target.

The levels of each tumor-associated mutation can be determined by quantifying each member of the first set of tumor-associated mutations and each member of the second set of tumor-associated mutations in circulating material isolated from the subject.

The circulating material can be any material generated by tumor or healthy cells of the subject and which can be found in the subject's bloodstream. Examples of circulating material include circulating tumor DNA (ctDNA), circulating free DNA (cfDNA), circulating tumor cells (CTCs), circulating tumor proteins, extracellular vesicles, or two or more thereof.

Circulating tumor DNA is DNA from tumor cells, and as such, are expected to contain tumor-associated mutations. Even if the tumor itself is growing, individual tumor cells can die and release ctDNA, either directly or through the normal action of macrophages.

The abundance of ctDNA is a function of the total area of tumor-blood vessel interface. This includes all metastases. ctDNA thus aggregates information from all of the subject's tumors, whereas quantifying tumor-associated mutations by WES, WGS, or scRNA sequencing of tissue instead of blood is limited to the particular tumors from which tissue is extracted for sequencing. Also, drawing of blood is a simpler and less invasive process than extracting tissue from most internal tumors.

Exemplary amounts of ctDNA in a biological sample (e.g., plasma or serum) can range from about 1 femtogram (fg) to about 1 picogram (pg), about 1 pg to about 200 nanogram (ng), about 1 ng to about 100 ng, or about 10 ng to about 1000 ng. For example, the amount can be up to about 600 ng, up to about 500 ng, up to about 400 ng, up to about 300 ng, up to about 200 ng, up to about 100 ng, up to about 50 ng, or up to about 20 ng of ctDNA. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of ctDNA. The amount can be up to 1 fg. 10 fg, 100 fg, 1 pg, 10 pg, 100 pg, 1 ng, 10 ng, 100 ng, 150 ng, or 200 ng of ctDNA molecules. The method can comprise obtaining 1 fg to 200 ng ctDNA.

The ctDNA can have an exemplary size distribution of about 100-500 nucleotides. The ctDNA can be about 100, about 105, about 110, about 115, about 120, about 125, about 130, about 135, about 140, about 145, about 150, about 155, about 160, about 165, about 170, about 175, about 180, about 185, about 190, about 195, about 200, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, or about 500 nucleotides.

In its broadest definition, circulating free DNA is any DNA present in the bloodstream. Herein, cfDNA is used to refer to DNA released into the bloodstream from non-tumor cells. In other words, ctDNA and cfDNA are distinct aspects of the present disclosure.

In addition to ctDNA, the bloodstream also contains circulating free DNA (cfDNA) from non-tumor sources, namely, normal cells. Generally, 1 ml of blood contains an average of 6 ng of circulating free DNA, or roughly 1800 cellular genomes. The concentration of cfDNA and ctDNA fluctuate widely between different individuals and is dependent on many factors including age, lifestyle, conditions, and in the context of cancer treatment, the type and stage of the cancer. Of these cellular genomes, the majority will be from healthy cells. For example, a breast cancer tumor with a diameter of 10 mm is expected to contribute about 0.03 cancer genomes/ml of blood. For another example, a melanoma tumor with a diameter of 10 mm is expected to contribute about 1-2 cancer genomes/ml of blood.

Even though cfDNA is DNA from non-tumor cells, and thus, would not at first glance seem likely to contain tumor-associated mutations, DNA from non-tumor cells present in the tumor microenvironment may undergo epigenetic changes, such as changes in methylation or changes in chromatin content, that reflect tumor-associated mutations.

Circulating tumor proteins are proteins from tumor cells, and as such, are expected to include tumor-associated mutations. Circulating tumor proteins can be released from dead or dying tumor cells, can be secreted by live tumor cells, or introduced into the subject's bloodstream by any other mechanism that may be known.

CTCs are tumor cells, whether live, dying, or dead, present in the bloodstream. Being cells, they contain tumor DNA and tumor proteins, and accordingly are expected to contain tumor-associated mutations.

Extracellular vesicles are bodies defined by membranes that can be released from the surface of tumor or normal cells. Extracellular vesicles typically contain RNA and proteins from the parent cell, which can provide information on tumor-associated mutations. For example, extracellular vesicles from tumor cells can be a source of tumor proteins, and any extracellular vesicle can be a source of nucleic acids indicative of epigenetic changes in a tumor cell or a normal cell that lead to changes in DNA transcription (and expected resultant changes in RNA levels) for one or more genes.

To quantify the tumor-associated mutations in circulating material, a sample containing circulating material can be taken from the subject, and circulating material can be isolated therefrom.

The sample can be obtained from human or non-human subjects. In one aspect, the sample is obtained from a human. The sample can be obtained from a variety of biological sources.

Various assays (e.g., sequencing assays) can be used to detect circulating material. The methods provided herein can comprise isolation and analysis of circulating material from the blood (e.g., plasma or serum) of a subject of interest (i.e., a subject having cancer, a subject in remission of cancer, or a subject suspected of having cancer). The method can comprise isolating plasma and circulating material from intact cell-depleted blood. The method can comprise centrifugation to generate plasma and extraction of nucleic acids from plasma.

The circulating material can be from a bodily fluid (e.g., a blood sample) of a subject of interest. The circulating material can be obtained from plasma fraction, serum fraction, or both, of the blood sample. In some embodiments, the bodily sample is whole blood, serum, plasma, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine, or any combination thereof. In some embodiments, the circulating DNA is obtained from blood and fractions thereof. A sample can be in the form originally isolated from a subject or can be subjected to further processing to remove or add components, such as cells, or enrich for one component relative to another. A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis. The subject can be a human, a mammal, an animal, a companion animal, a service animal, or a pet. The subject may not have cancer or a detectable cancer symptom. The subject may have been treated with one or more cancer therapy, e.g., any one or more of chemotherapies, antibodies, vaccines, or biologics. The subject may be in remission. The subject may be suspected to have cancer or any cancer-associated genetic mutations.

The biological sample can be obtained from a subject by any means including, but not limited to, tumor biopsy, needle aspirate, scraping, surgical excision, surgical incision, venipuncture, or other means known in the art. Those skilled in the art will recognize other suitable techniques for obtaining biological samples.

The biological sample can be obtained from the subject in a single procedure. The biological sample can be obtained from the subject repeatedly over a period of time. For example, the biological sample may be obtained once a day, once a week, monthly, biannually, or annually. Obtaining numerous samples over a period of time can be useful to identify and select new tumor-specific neoantigens.

The circulating material can be isolated from bodily fluids (e.g., plasma) through a fractionation or partitioning step in which circulating material, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, nucleic acids can be precipitated with an alcohol. Further clean up steps may be used such as silica based columns to remove contaminants or salts. After such processing, samples can include various forms of nucleic acid including double stranded DNA and single stranded DNA. In some embodiments, single stranded DNA can be converted to double stranded forms so they are included in subsequent processing and analysis steps.

In some embodiments, the circulating material is isolated from a blood sample, a serum sample, a plasma sample, a urine sample, or a cerebrospinal fluid sample. In one aspect, the sample is a blood sample, a serum sample, or a plasma sample.

In embodiments wherein the sample is a blood sample, a serum sample, or a plasma sample, the circulating material can be isolated from at least about 10 ml of the subject's whole blood, such as at least about 20 ml of the subject's whole blood. These blood volumes can be of particular utility when the circulating material comprises ctDNA, in view of the relatively small amount of cancer genomes/ml of blood.

In some embodiments, for ctDNA, a tumor-associated mutation can be found that has an allele frequency in the cfDNA pool of less than 10.0%, such as less than 9.9%, less than 9.8%, less than 9.7%, less than 9.6%, less than 9.5%, less than 9.4%, less than 9.3%, less than 9.2%, less than 9.1%, less than 9.0%, less than 8.9%, less than 8.8%, less than 8.7%, less than 8.6%, less than 8.5%, less than 8.4%, less than 8.3%, less than 8.2%, less than 8.1%, less than 8.0%, less than 7.9%, less than 7.8%, less than 7.7%, less than 7.6%, less than 7.5%, less than 7.4%, less than 7.3%, less than 7.2%, less than 7.1%, less than 7.0%, less than 6.9%, less than 6.8%, less than 6.7%, less than 6.6%, less than 6.5%, less than 6.4%, less than 6.3%, less than 6.2%, less than 6.1%, less than 6.0%, less than 5.9%, less than 5.8%, less than 5.7%, less than 5.6%, less than 5.5%, less than 5.4%, less than 5.3%, less than 5.2%, less than 5.1%, less than 5.0%, less than 4.9%, less than 4.8%, less than 4.7%, less than 4.6%, less than 4.5%, less than 4.4%, less than 4.3%, less than 4.2%, less than 4.1%, less than 4.0%, less than 3.9%, less than 3.8%, less than 3.7%, less than 3.6%, less than 3.5%, less than 3.4%, less than 3.3%, less than 3.2%, less than 3.1%, less than 3.0%, less than 2.9%, less than 2.8%, less than 2.7%, less than 2.6%, less than 2.5%, less than 2.4%, less than 2.3%, less than 2.2%, less than 2.1%, less than 2.0%, less than 1.9%, less than 1.8%, less than 1.7%, less than 1.6%, less than 1.5%, less than 1.4%, less than 1.3%, less than 1.2%, less than 1.1%, less than 1.0%, less than 0.9%, less than 0.8%, less than 0.7%, less than 0.6%, less than 0.5%, less than 0.4%, less than 0.3%, less than 0.2%, or less than 0.1%.

In some embodiments, blood samples can be collected at a minimum of once pre-treatment, and followed by serial collections guided by the monitoring-frequency of the immunogenic composition efficacy and disease progression.

Before treatment, 10-20 ml of blood can be collected from a subject within a 5-day interval of the biopsy collected for paired genomic (including, but not limited to, WGS/WES) tumor-normal sequencing, and within 2-4 weeks before the immunogenic composition is administered.

After treatment, serial collection of blood specimens can be performed based on medically informed indications of disease progression, clinically observed response to the immunogenic composition, or at regularly scheduled intervals when patients are scheduled to get follow-up doses of the initial immunogenic composition or a reformulated immunogenic composition, and at a minimum of once at the time of initial immunogenic composition administration and once within 10 weeks of initial immunogenic composition administration.

Quantifying the tumor-associated mutations can involve various methods depending on the circulating material under consideration. Quantifying can comprise sequencing ctDNA using WES, WGS, targeted sequencing, polymerase chain reaction (PCR), or hybridization methods.

For cfDNA, quantifying the tumor-associated mutations can comprise quantitative polymnerase chain reaction (qPCR) or next generation sequencing.

Tumor-associated mutations in nucleic acids from CTCs or extracellular vesicles can be quantified by quantified by any of the techniques described herein, bearing in mind that a reverse transcription process (e.g., use of a reverse transcriptase enzyme) can be used to prepare DNA from an RNA present in a CTC or an extracellular vesicle. Also, RNA can be directly subjected to single-cell RNA sequencing (scRNAseq) to identify and quantify tumor-associated mutations from RNA.

For any genomic DNA-based methods, such as for ctDNA, cfDNA, or DNA from CTCs, quantifying the tumor-associated mutations can comprise assaying methylation or chromatin content for one or more epigenetic markers. Chromatin content can be assayed by Assay for Transposase-Accessible Chromatin with High-Throughput Sequencing (ATAC-Seq), among other techniques known to a person of ordinary skill in the art.

A variety of methods exist for obtaining the genome sequence data described herein. Sequencing methods are well known in the art and include, but are not limited to, WGS, WES, targeted sequencing, PCR-based methods, including real-time PCR (RT-PCR), deep sequencing, high-throughput sequencing, or combinations thereof. In some instances, the foregoing techniques and procedures can be performed according to the methods described in e.g., Sambrook et al., Molecular Cloning: A Laboratory Manual 4th ed. (2012) Cold Spring Harbor Laboratory Press, Cold Spring Harbor, NY. See also, Austell et al., Current Protocols in Molecular Biology, ed., Greene Publishing and Wiley-Interscience New York (1992) (with periodic updates).

The genome sequence data can be obtained from WGS, WES, targeted sequencing, DNA hybridization methods or combinations thereof. The genome sequencing data can be sequence data derived from high-depth WGS data.

In some embodiments, DNA can be sequenced to obtain sequence reads. The sequence data comprises sequence reads of a plurality of polynucleotides from the subject. Sequence reads can comprise about 2 to about 5000 nucleotides. For example, about 2, about 3, about 4, about 5, about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 550, about 600, about 700, about 800, about 900, about 1,000, about 1,100, about 1,200, about 1,300, about 1,400, about 1,500, about 1,600, about 1,700, about 1,800, about 1,900, about 2,000, about 2,100, about 2,200, about 2,300, about 2,400, about 2,500, about 2,600, about 2,700, about 2,800, about 2,900, about 3,000, about 3,100, about 3,200, about 3,300, about 3,400, about 3,500, about 3,600, about 3,700, about 3,800, about 3,900, about 4,000, about 4,100, about 4,200, about 4,300, about 4,400, about 4,500, about 4,600, about 4,700, about 4,800, about 4,900, or about 5,000 nucleotides.

DNA sequence data can be analyzed using integration of variant reads (INVAR) as described in International Application No.: PCT/EP2019/055610. The DNA sequence data can be analyzed using an objective function outlined in Equation 1:

$$N = {\sum }_{i = 1}^{C} {ξ}_{i} {⋁}_{α = 1}^{L} {\tilde{P}}_{i α} {s}_{α} \qquad Eq . 1$$

where ξi is the ith sub-clone's estimated cellular prevalence. P˜iαis the probability that peptide α is in the ith sub-clone, and su is the individual score of the alpha'th peptide from the machine learning modeling. All of these prevalence and sub-clonality estimates are derived from the combination of biopsy and DNA sequencing. The “big-OR” is taken over the best L peptides, with L=20, and the summation is taken over all of the C sub-clones, with C a parameter returned by the sub-clonality estimation algorithms. Typically 1<C<15.

The DNA sequence data can be analyzed using an objective function outlined in Equation 2:

$$N = {\sum }_{α = 1}^{L} {ξ}_{α} {s}_{α} \qquad Eq . 2$$

where ξα is the cellular prevalence of the alpha'th mutation and sα is the individual score of the alpha'th peptide from the machine learning modeling. A straight sort of all peptides by the product ξαsα optimizes this function.

The DNA sequence data can be analyzed using a machine learning platform.

Exemplary machine learning models that can be suitable include, but are not limited to, a neural network, a Bayesian classifier, a logistic regression, a decision tree, a gradient boosting decision tree, a random forest, a support vector machine, a gradient-boosted tree, a multilayer perceptron, a one-vs-rest, or a Gaussian Naive Bayes.

In some embodiments, blood samples can be processed within 4 hours and circulating free DNA can be isolated from plasma using standard methods or kits (e.g., DNeasy™ kit (Qiagen N.V., Venlo, Netherlands), QIAmp™ kit (Id.), or Quick-cfDNA™ kit (Zymo Research, Irvine, CA)). In the pre-treatment specimen processing, separate from the circulating free DNA isolation from plasma, peripheral blood lymphocytes from the first centrifugation step in circulating free DNA isolation can also be used to extract germline genomic DNA, which can be used as the matched normal for genomic analysis. A minimum yield of 20 ng of DNA per blood aliquot is desirable.

As described herein, the circulating material can comprise circulating tumor proteins, proteins from CTCs, or proteins from extracellular vesicles, any of which may comprise tumor-associated mutations. Quantifying tumor-associated mutations in proteins can comprise performing mass spectrometry or elution assays on circulating tumor proteins, proteins from CTCs, or proteins from extracellular vesicles.

As described herein, CTCs can be a source of tumor proteins or tumor nucleic acids. Also, quantifying of tumor-associated mutations in material from CTCs can comprise performing fluorescence-activated cell sorting (FACS) on CTCs. This can be part of an initial classification or separation of CTCs from other cells in the circulation. In some embodiments, particularly wherein a tumor-associated mutation is in a protein exposed on the surface of tumor cells, FACS can be used to sort CTCs based at least in part on a tumor-associated mutation, and hence can provide direct quantification of the tumor-associated mutation.

The units of quantification can be molecules/ml or another metric of tumor-associated mutation copy number per weight or volume of sample. The quantification can be normalized if desired, such as by normalizing tumor-associated mutation copy number to the total amount of the source material, number of copies of a healthy tissue marker gene that is expected to have a constant copy number in the source material, or the like.

The quantification of tumor-associated mutations can be performed at each of multiple time points. By doing so, the quantity of each tumor-associated mutation can be tracked over time.

The quantification of a tumor-associated mutation can be performed using the same circulating material at each time point. For one hypothetical example, if a first tumor-associated mutation is quantified from ctDNA at a first time point, it can be quantified from ctDNA at a second time point. This may be desirable to provide greater consistency and accuracy of measurements over multiple time points.

Contemplated is the quantification of a first tumor-associated mutation from a first circulating material at a first time point and a second circulating material at a second time point. For another hypothetical example, a first tumor-associated mutation can be quantified from ctDNA at a first time point and from CTCs at a second time point. With appropriate normalization of the quantified values determined by each technique to reference values that are expected to remain both substantially constant within each circulating material and substantially proportional between the two circulating materials over the various time points, the accuracy of measurements over multiple time points may be maintained at an acceptably high level.

Multiple circulating materials can be used to quantify multiple tumor-associated mutations. For yet another hypothetical example, a first tumor-associated mutation can be quantified from ctDNA at a first time point and a second time point, and a second tumor-associated mutation can be quantified from e.g., circulating tumor proteins at the first time point and the second time point.

In some embodiments, at least one of the multiple time points can be before the initial immunogenic composition is administered.

The hypothetical model illustrated in FIG. 1 shows quantification of three tumor-specific neoantigens, peptides 1-3, from ctDNA. As discussed above, peptides 1 and 2 are included in an immunogenic composition and correspond to members of a first set of tumor-associated mutations. Peptide 3 is not included in the immunogenic composition and corresponds to a member of a second set of tumor-associated mutations. The three tumor-associated mutations corresponding to peptides 1-3 are quantified in ctDNA at four time points, week E, week 0, week 4, and week 8. Week E is a time point at which the subject's tumor-associated mutations are identified. An immunogenic composition, referred to in FIG. 1 as a vaccine, is manufactured prior to week 0. The immunogenic composition is administered to the subject at week 0, week 4, and week 8, as represented by the notation “VAC.” Thus, at least one time point of quantifying, week E, is before the initial immunogenic composition is administered.

Continuing the hypothetical model of FIG. 1, the quantities of each tumor-associated mutation are graphed over time. After week 8. the following are observed:

The quantity of the tumor-associated mutation corresponding to peptide 1, comprising ProTrp, has declined over time. This indicates that the tumor-specific neoantigen of peptide 1 in the initial immunogenic composition has suppressed tumor cells containing this tumor-associated mutation;

The quantity of the tumor-associated mutation corresponding to peptide 2, comprising ArgCys, has increased over time. This indicates that the tumor-specific neoantigen of peptide 2 in the initial immunogenic composition has failed to suppress tumor cells containing this tumor-associated mutation;

The quantity of the tumor-associated mutation corresponding to peptide 3, comprising ThrAla, has slightly increased over time. However, it should be bome in mind that the tumor-specific neoantigen of peptide 3 was not included in the initial immunogenic composition.

The multiple time points at which the tumor-associated mutations are quantified can occur at various intervals and/or at different times relative to other actions according to the method, as will be discussed in more detail herein.

### E. Reformulation of Immunogenic Compositions

By quantifying tumor-associated mutations in circulating material over multiple time points, it may be discovered that the quantity of a member of the first set of tumor-associated mutations (i.e., a tumor-associated mutation which has a corresponding tumor-specific neoantigen included in the initial immunogenic composition) increases from an earlier time point to a later time point. As discussed above, such a discovery indicates the tumor-specific neoantigen corresponding thereto is an ineffective component of the initial immunogenic composition.

The methods can further comprise replacing a tumor-specific neoantigen corresponding to a member of the first set of tumor-associated mutations from the initial immunogenic composition with a replacement tumor-specific neoantigen corresponding to a member of the second set of tumor-associated mutations, to yield a reformulated immunogenic composition, in response to the quantity of the member of the first set of tumor-associated mutations increasing from an earlier time point to a later time point.

As should be apparent, multiple tumor-specific neoantigens, each corresponding to a member of the first set of tumor-associated mutations, can be replaced, if the condition is met that the quantity of the member of the first set of tumor-associated mutations corresponding to that tumor-specific neoantigen increased from an earlier time point to a later time point.

In some embodiments, the quantity of the member of the second set of tumor-associated mutations to which the replacement tumor-specific neoantigen corresponds did not decrease from the earlier time point to the later time point. This can be desirable, on the assumption that if the quantity of the tumor-associated mutation in question decreased, even if the tumor-specific neoantigen to which it corresponds was not targeted by the immunogenic composition, then there would be no further benefit to targeting it with a replacement tumor-specific neoantigen.

The members of the second set of tumor-associated mutations can be changed if the method is performed over multiple iterations. This can involve periodic monitoring of the subject's circulating material for the emergence of new neoantigens. When anew neoantigen emerges and is detected, the sequence encoding it can be added to the second set of tumor-associated mutations.

In some embodiments, the method can further comprise detecting the emergence at a first time point of at least one tumor-associated mutation not included in the first set of tumor-associated mutations or the second set of tumor-associated mutations; and adding the emerged tumor-associated mutation to the second set of tumor-associated mutations at a time point after the first time point. After the addition of the emerged tumor-associated mutation to the second set, at additional time points, the immunogenic composition can be reformulated to include the emerged tumor-associated mutation.

As a particular hypothetical example, an initial immunogenic composition can be prepared based in part on a finding that a peptide p is not encoded by the subject's ctDNA. In other words, peptide p does not arise from a tumor-associated mutation and thus is not suitable for inclusion in the initial immunogenic composition. At a first time point (e.g., after at least one round of quantifying members of the second set of tumor-associated mutations and monitoring for the emergence of a new neoantigens), a new neoantigen is detected. The new neoantigen can be added to the second set of tumor-associated mutations and the method continued to a later time point. If the quantity of the new neoantigen does not decrease over two or more time points, it may be a highly desirable reformulation candidate, because the new neoantigen may reflect dynamic responses of the tumor to the treatment (e.g., a new subclone carrying the new neoantigen may have gained resistance to all the components in the initial immunogenic composition). Reformulating the composition to include the new neoantigen may minimize the subclone's ability to escape treatment.

The replacement tumor-specific neoantigen can be selected based on any one or more criteria that will be apparent to a person of ordinary skill in the art. In some embodiments, the tumor-specific neoantigens corresponding to the second set of tumor-associated mutations can be ranked by immunogenicity score and the highest-ranking tumor-specific neoantigen can be selected as the replacement tumor-specific neoantigen. In some embodiments, the changes in the quantities of the second set of tumor-associated mutations over the multiple time points can be ranked, and the tumor-specific neoantigen corresponding to the tumor-associated mutation with the largest increase can be selected as the replacement tumor-specific neoantigen.

The replacing can be performed at any time after administration of the initial immunogenic composition. In some embodiments, the initial immunogenic composition can be administered once before the replacing of the tumor-specific neoantigen.

In some embodiments, the initial immunogenic composition can be administered multiple times before the replacing of the tumor-specific neoantigen.

Returning to the hypothetical model of FIG. 1, the initial immunogenic composition is administered three times (at week 0, week 4, and week 8). Thereafter, the tumor-associated mutation from the first set of tumor-associated mutations (those with corresponding tumor-specific neoantigens that were included in the initial immunogenic composition) that increased in quantity from week E to week 8, which was peptide 2, comprising ArgCys, is replaced in the immunogenic composition with peptide 3, comprising ThrAla. Although not required in the present method, the quantity of the tumor-associated mutation to which peptide 3 corresponds did not decrease from week E to week 8. The resulting reformulated immunogenic composition comprises peptide 1 (comprising ProTrp) and peptide 3 (comprising ThrAla). The reformulated immunogenic composition can be administered at week 12, week 16, and week 20. In other words, the regimen of immunogenic composition administration can remain the same, except that the reformulated immunogenic composition instead of the initial immunogenic composition is administered at scheduled administration times after replacing the prior tumor-specific neoantigen with the replacement tumor-specific neoantigen.

The replacement immunogenic composition can be administered at any time after the replacing. In some embodiments, the reformulated immunogenic composition is administered once after replacing the tumor-specific neoantigen.

In some embodiments, the reformulated immunogenic composition can be administered multiple times after replacing the tumor-specific neoantigen.

### F. Selection of Time Points for Quantifying

As discussed above, in some embodiments, at least one of the multiple time points for quantifying can be chosen relative to the timing of other actions according to the method.

In some embodiments, one of the multiple time points is at least about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks after administering the initial immunogenic composition or the reformulated immunogenic composition.

In some embodiments, one of the multiple time points is at least about 1 month, about 2 months, about 3 months, about 4 months, about 5 months, about 6 months, about 7 months, about 8 months, about 9 months, about 10 months, about 11 months, or about 12 months after administering the initial immunogenic composition or the reformulated immunogenic composition.

In some embodiments, one of the multiple time points is at least about 1 year, about 2 years, about 3 years, about 4 years, about 5 years, about 6 years, about 7 years, about 8 years, about 9 years, or about 10 years after administering the initial immunogenic composition or the reformulated immunogenic composition.

G. Treatment Regimens Comprising More than Two Reformulated Immunogenic Compositions

The foregoing discussion of an initial immunogenic composition and a reformulated immunogenic composition is not limited to situations such as the hypothetical model depicted in FIG. 1 in which the initial immunogenic composition is the first immunogenic composition administered to the subject and the reformulated immunogenic composition is the second and final immunogenic composition administered to the subject. More generally, the immunogenic composition referred to herein as the initial immunogenic composition can be the xth immunogenic composition of n immunogenic compositions administered to the subject during the course of a treatment regimen, wherein x is from 1 to n−1, inclusive, and the reformulated immunogenic composition is the x+1th of n immunogenic compositions administered to the subject, wherein x+1 is from 2 to n, inclusive. This definition encompasses the hypothetical model of FIG. 1, wherein n=2, along with embodiments wherein n=3, 4, 5, 6, or more.

Accordingly, in some embodiments, a method disclosed herein can be performed multiple times during the course of a treatment regimen. For example, if n=3, a method disclosed herein can be performed twice. In the first performance, the first immunogenic composition administered during the treatment regimen is a first initial immunogenic composition and the second immunogenic composition administered during the treatment regimen is a first reformulated immunogenic composition. In the second performance, the first reformulated immunogenic composition is also a second initial immunogenic composition, and the third immunogenic composition administered during the treatment regimen is a second reformulated immunogenic composition.

### 4. EQUIVALENTS

It will be readily apparent to those skilled in the art that other suitable modifications and adaptions of the methods of the invention described herein are obvious and may be made using suitable equivalents without departing from the scope of the disclosure or the embodiments.

1. A method, comprising
administering to a subject in need thereof an initial immunogenic composition comprising a plurality of tumor-specific neoantigens, wherein each tumor-specific neoantigen corresponds to a member of a first set of tumor-associated mutations in the subject, and no tumor-specific neoantigen corresponds to a member of a second set of tumor-associated mutations in the subject; and
quantifying each member of the first set of tumor-associated mutations and each member of the second set of tumor-associated mutations in circulating material comprising tumor-associated mutations isolated from the subject at each of multiple time points.

2-6. (canceled)

7. The method of claim 1, wherein the circulating material comprises circulating tumor DNA (ctDNA), circulating free DNA (cfDNA), circulating tumor cells (CTCs), circulating tumor proteins, extracellular vesicles, or a combination thereof.

8. The method of claim 1, further comprising
replacing a tumor-specific neoantigen corresponding to a member of the first set of tumor-associated mutations from the initial immunogenic composition with a replacement tumor-specific neoantigen corresponding to a member of the second set of tumor-associated mutations, to yield a reformulated immunogenic composition, in response to the quantity of the member of the first set of tumor-associated mutations increasing from an earlier time point to a later time point; and
administering the reformulated immunogenic composition to the subject.

9. The method of claim 1, wherein the subject has melanoma, breast cancer, sarcomas, ovarian cancer, prostate cancer, kidney cancer, gastric cancer, colon cancer, testicular cancer, head and neck cancer, pancreatic cancer, brain cancer, bone cancer, B-cell lymphoma, acute myelogenous leukemia, chronic myelogenous leukemia, chronic lymphocytic leukemia, T-cell lymphocytic leukemia, colon cancer, urothelial cancer, or lung cancer.

10. The method of claim 1, wherein the tumor-associated mutations comprise at least one mutation specific to the subject.

11. The method of claim 1, wherein the tumor-associated mutations comprise at least one tumor hotspot mutation.

12. The method of claim 11, wherein the tumor is ER+/HER2− breast cancer and the at least one tumor hotspot mutation is in a gene selected from the group consisting of AKT1, APC, ARID1A, ATM, BRAF, BRCA1, BRCA2, CDH1, CDKN2A, ESR1, GATA3, GNAS, HER2, KRAS, NF1, PIK3CA, PTEN, RB1, SMAD4, and TP53.

13. The method of claim 11, wherein the tumor is melanoma.

14. The method of claim 1, wherein each tumor-specific neoantigen corresponding to a member of the first set of tumor-associated mutations has a higher immunogenicity score than any tumor-specific neoantigen corresponding to a member of the second set of tumor-associated mutations.

15. The method of claim 1, wherein at least one of the multiple time points is before the initial immunogenic composition is administered.

16. The method of claim 8, wherein the initial immunogenic composition is administered multiple times prior to the step of replacing the tumor-specific neoantigen.

17. The method of claim 8, wherein the reformulated immunogenic composition is administered multiple times after the step of replacing the tumor-specific neoantigen.

18. The method of claim 8, wherein the quantity of the member of the second set of tumor-associated mutations to which the replacement tumor-specific neoantigen corresponds did not decrease from the earlier time point to the later time point.

19. The method of claim 7, wherein quantifying the tumor-associated mutations comprises
sequencing ctDNA using whole exome sequencing (WES), whole genome sequencing (WGS), targeted sequencing, polymerase chain reaction (PCR), or hybridization methods;
sequencing cfDNA using quantitative polymerase chain reaction (qPCR) or next generation sequencing;
assaying methylation or chromatin content of ctDNA, cfDNA, or DNA from CTCs;
performing mass spectrometry or elution assays on circulating tumor proteins, proteins from CTCs, or proteins from extracellular vesicles;
performing fluorescence-activated cell sorting (FACS) on CTCs; or
sequencing nucleic acids from CTCs or extracellular vesicles using WES, WGS, targeting sequencing, PCR, qPCR, next generation sequencing, single-cell RNA sequencing, or hybridization methods.

20. The method of claim 8, wherein one of the multiple time points is at least about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks after administering the initial immunogenic composition or the reformulated immunogenic composition.

21. The method of claim 8, wherein one of the multiple time points is at least about 1 month, about 2 months, about 3 months, about 4 months, about 5 months, about 6 months, about 7 months, about 8 months, about 9 months, about 10 months, about 11 months, or about 12 months after administering the initial immunogenic composition or the reformulated immunogenic composition.

22. The method of claim 8, wherein one of the multiple time points is at least about 1 year, about 2 years, about 3 years, about 4 years, about 5 years, about 6 years, about 7 years, about 8 years, about 9 years, or about 10 years, after administering the initial immunogenic composition or the reformulated immunogenic composition.

23. The method of claim 1, wherein the circulating material is isolated from a blood sample, a serum sample, a plasma sample, a urine sample, or a cerebrospinal fluid sample.

24. The method of claim 1, wherein the circulating material is isolated from at least about 10 ml of the subject's whole blood.

25. (canceled)

26. The method of claim 1, further comprising
detecting the emergence at a first time point of at least one tumor-associated mutation not included in the first set of tumor-associated mutations or the second set of tumor-associated mutations; and
adding the emerged tumor-associated mutation to the second set of tumor-associated mutations at a time point after the first time point.
